(19) United States
(12) Patent Application Publication
(10) Pub. No.: US 2011/0098509 A1
(43) Pub. Date: Apr. 28, 2011

(54) PROCESS FOR THE PREPARATION OF PHENOL BY MEANS OF NEW CATALYTIC SYSTEMS

(75) Inventors: Francesco Minisci, Milan (IT); Ombretta Porta, Milan (IT); Angelo Clerici, Milan (IT); Alberto Clerici, Milan (IT); Francesco Recupero, Milan (IT); Carlo Punta, Isola Del Cantone (Genova) (IT); Cristian Gambarotti, Cremona (IT); Monica Pierini, Melegnano (Milan) (IT)

(73) Assignee: POLIMERI EUROPA S.p.A., San Donato Milanese (Milan) (IT)

(21) Appl. No.: 12/443,271

(22) PCT Filed: Sep. 20, 2007
(86) PCT No.: PCT/EP2007/008341

§ 371 Date: Nov. 18, 2010

(30) Foreign Application Priority Data
Sep. 28, 2006 (IT) MI2006A001859

(51) Int. Cl. C07C 407/00; C07C 37/08

(52) U.S. Cl. 568/577; 568/798

(57) ABSTRACT

The invention relates to a process for the preparation of phenol which comprises the aerobic oxidation of cumene to hydroperoxide with high conversions and selectivities, in the presence of new catalytic systems, extremely mild conditions and the subsequent acid decomposition of the hydroperoxide to phenol and acetone.

The present invention relates to a process for the preparation of phenol by the aerobic oxidation of cumene which is based on the use of a new catalytic system.

More specifically, the invention relates to a process for the preparation of phenol by the aerobic oxidation of cumene and subsequent acid decomposition of hydroperoxide to phenol and acetone, carried out in the presence of new catalytic systems, under extremely mild conditions and with high conversions and selectivities.

The Hock process for the production of phenol, commonly used by the chemical industry, is based on the auto-oxidation of cumene to hydroperoxide, which is then decomposed by means of acid catalysis to phenol and acetone (H. Hock, S. Lang, Ber. 1944, 77, 257; W. Jordan, H. Van Barmeveld, O. Gerlich, M. K. Baymann, S. Ulrich, Ullman's Encyclopedia of Industrial Organic Chemicals, Vol. A 9, Wiley-VCH, Weinheim, 1985, 299).

The most critical aspect of the process is the auto-oxidation phase which is characterized by a classical radical chain process in which the hydroperoxide formed acts in turn as initiator of the radical chain. The selectivity in the formation of the hydroperoxide decreases to the extent in which the hydroperoxide itself acts as initiator as its decomposition produces acetophenone, which is the main by-product at relatively high temperatures, and cumyl alcohol. The decomposition of the hydroperoxide, on the other hand, increases with the conversion (the greater the conversion and therefore the concentration of hydroperoxide, the higher the decomposition will be) and with the temperature. The lower the conversion and temperature, the higher the formation selectivity of hydroperoxide will be.

Another important aspect is the necessity, for industrial processes, of operating in an alkaline environment in order to neutralize the carboxylic acids, essentially formic acid, which are formed during the oxidation and which catalyze the decomposition of the hydroperoxide to phenol which is an auto-oxidation process inhibitor.

At temperatures lower than 100° C., the non-catalyzed oxidation of cumene is too slow; upon increasing the temperature, the conversion increases, but the selectivity decreases. In any case, the conversion of cumene cannot be high as the consequent selectivity is considerably jeopardized.

Under industrial conditions for the non-catalyzed peroxidation of cumene, a compromise between temperature, conversion and selectivity has always been sought.

The use of metallic salts (Co, Mn) as catalysts considerably increases the aerobic oxidation rate of the cumene and allows lower temperatures to be used, but it also significantly reduces the selectivity as these metallic salts accelerate the decomposition of the hydroperoxide.

This type of catalysis does not seem particularly suitable for the production of cumene hydroperoxide by means of aerobic oxidation (F. Minisci, F. Recupero, A. Cecchetto, C. Gambarotti, C. Punta, R. Paganelli Org. Proc. Res. Devel. 2004, 163).

A different approach concerns the use as catalysts of N-hydroxyphthalimide both in association with cumene hydroperoxide (R. A. Sheldon, I.W.E. Arends Adv. Synth. Catal. 2001, 343, 1051) and with traditional radical initiators, such as azoisobutyronitrile (O. Fueuda, S. Sakaguchi, Y. Ishii Adv. Synth. Catal. 2001, 343, 809).

Also in these cases, temperatures ranging from 75° C. to 100° C. are used; either the conversions or the selectivities are not high; moreover the N-hydroxyphthalimide is decomposed during the oxidation. At lower temperatures, these initiators are not effective. It is not possible in these cases to use solvents, such as acetic acid, which, at the oxidation temperature, partially decompose the cumene hydroperoxide to phenol, inhibiting the auto-oxidation process itself.

A catalytic system has now been found, which allows the aerobic oxidation of cumene to be carried out under particularly mild temperature and pressure conditions. Furthermore, this catalytic system allows high conversions to be obtained, associated with high selectivities, unlike the industrial processes currently in use, in which the selectivities decrease with an increase in the conversions.

An object of the present invention therefore relates to a process for the preparation of cumene hydroperoxide characterized in that cumene is reacted with oxygen in the presence of a catalytic system comprising an N-hydroxyimide or an N-hydroxysulfonamide having general formula I and II,

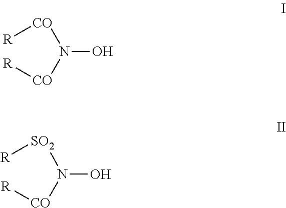

wherein R is an alkyl, aryl group or is part of aliphatic and aromatic cyclic systems, associated with a peracid or dioxirane, at a temperature<100° C.

The N-hydroxyimide or N-hydroxysulfonamide is preferably selected from the group consisting of N-hydroxysuccinimide, N-hydroxyphthalimide, N-hydroxysaccharine.

N-hydroxyphthalimide and N-hydroxysuccinimide are of particular industrial interest, as they are easily accessible from low-cost industrial products such as phthalic or succinic anhydride.

A further object of the present invention relates to a process for the preparation of phenol which comprises the preparation of cumene hydroperoxide as previously described and the subsequent acid decomposition of the hydroperoxide to phenol and acetone.

In any case, the N-hydroxy-derivatives are not decomposed due to the particularly mild conditions of the oxidation process and can be recovered and recycled, contrary to what occurs when the same derivatives are used at higher temperatures.

The peracids and dioxiranes can be either aliphatic or aromatic commercial products, such as peracetic or m-chloroperbenzoic acid, whereas the dioxiranes are prepared starting from ketones and potassium monopersulfate (A. Bravo, F. Fontana, G. Fronza, F. Minisci J. Org. Chem. 1998, 63, 254).

Instead of peracids or dioxiranes, precursors such as aldehydes for the peracids and a mixture of ketones and potassium monopersulfate for the dioxiranes, can be used more economically.

The use of aldehydes, such as acetaldehyde or benzaldehyde, is particularly convenient, as, under the reaction conditions, they are slowly oxidized to peracids by oxygen, and do not require further oxidizing agents, as in the case of dioxiranes.

This relatively slow oxidation process of aldehydes is useful as, given the same conditions, the conversions of cumene increase maintaining low stationary concentrations of peracid.

An analogous result can be obtained by slowly adding the peracid or dioxirane to the reaction mixture as the peracids and dioxiranes are decomposed during the oxidation to cumene, maintaining their stationary concentrations low, whereas the N-hydroxy-derivatives remain unaltered and can be recycled.

In order to trigger the oxidation of aldehydes and reduce the induction period, it is also possible to use a very small quantity of peracid.

The oxidation can be carried out with cumene in a solution of solvents such as acetonitrile, acetone, dimethylcarbonate or ethylacetate, which do not easily form explosive mixtures with oxygen under mild conditions; the latter also allow the use of acetic acid as solvent, with which it is even more difficult to form explosive mixtures with oxygen as, under the mild conditions used, the acetic acid does not catalyze the decomposition of hydroperoxide to phenol. In all the other processes described and mentioned above, the acetic acid inhibits the oxidation process and cannot be used as solvent. It is also possible to operate without solvents, but in this case an N-hydroxy-derivative must be used, which is soluble in cumene as the simplest chain-ends (N-hydroxysuccinimide, N-hydroxyphthalimide, N-hydroxysaccharine) are not very soluble. The solubility of the N-hydroxy-derivative in cumene is increased by introducing sufficiently long alkyl chains (C₆-C₁₄ into the N-hydroxy-derivative itself.

The hydroperoxide solution is decomposed to phenol or acetone by means of homogeneous or heterogeneous catalysis; the latter, obtained by the use of acid polymers such as Amberlyst 15 or Nafion, is particularly advantageous for the isolation of the phenol and recycling of the catalyst after separation.

The oxidation is carried out at temperatures lower than 100° C. and preferably at atmospheric pressure. It is preferably carried out at temperatures ranging from 20° C. to 70° C.

Quantities of N-hydroxy-derivatives, peracids or dioxiranes ranging from 1 to 10% with respect to the cumene, are preferably used; when the N-hydroxy-derivative is associated with an aldehyde the quantity of the latter preferably ranges from 1% to 20% with respect to the cumene.

An important discovery is that neither N-hydroxy-derivatives, nor peracids, or dioxiranes or their precursors alone have catalytic activities in the aerobic oxidation of cumene under the particularly mild operating conditions used; i.e. a significant oxidation does not take place using an N-hydroxy-derivative or peracid or dioxirane or one of their precursors alone as catalysts.

Under the operating conditions adopted, cumene hydroperoxide or other hydroperoxides, such as azoisobutyronitrile or benzoylperoxide, in association with N-hydroxy-derivatives, are completely inert and have no initiation activity of aerobic oxidation processes of cumene. This is contrary to the industrial oxidation processes currently adopted in which, operating at high temperatures, the initiation of the oxygenation process occurs by the thermal decomposition of the cumene hydroperoxide, which therefore reduces the process selectivity as the conversion and consequently the concentration of hydroperoxide increase.

This explains the possibility of obtaining, under mild temperature conditions, high conversions associated with high selectivities by means of the new catalytic systems discovered with this invention.

This result is due to a different operating mechanism of peracids and dioxiranes which are stable at the reaction temperatures without N-hydroxy-derivates and consequently do not initiate oxidation processes by means of thermal decomposition, with respect to the use of initiators such as cumene hydroperoxide or azoisobutyronitrile used formerly, which are inert at a low temperature and must be brought to decomposition temperatures to be able to initiate and maintain the oxidation process of cumene.

With the catalysts of this invention, the initiation and maintenance of the oxidation process of cumene occur as a result of the reaction, even at low temperatures, between N-hydroxy-derivatives and peracids or dioxiranes, which are separately stable under these conditions.

The following examples are provided for illustrative purposes but in no way limit the process object of the present invention.

### EXAMPLE 1

A solution of 2.5 mmoles of m-chloroperbenzoic acid in 10 mL of acetonitrile is added dropwise under stirring to a solution of 50 mmoles of cumene and 5 mmoles of N-hydroxyphthalimide in 100 mL of acetonitrile, in an oxygen atmosphere, at atmospheric pressure, at 20° C. over a period of 12 hours. HPLC analysis of the reaction mixture shows a conversion of cumene of 91% with a yield of cumyl-hydroperoxide of 97% based on the cumene converted, whereas the N-hydroxyphthalimide remains substantially unaltered. The reaction mixture is treated with a 0.3 M solution of H₂SO₄ in acetonitrile (5 mL) for 2 hours at room temperature, obtaining phenol with a yield of 92% with respect to the cumene converted.

### EXAMPLE 2

The same procedure is effected as in Example 1 without m-chloroperbenzoic acid. There is no significant oxidation.

### EXAMPLE 3

The same procedure is effected as in Example 1 without N-hydroxyphthalimide; the conversion of cumene is 1% with the formation of traces of cumyl alcohol.

### EXAMPLE 4

The same procedure is effected as in Example 1 in which all the m-chloroperbenzoic acid was added to the reaction mixture at the beginning. The cumene conversion is 70% with a yield to cumyl-hydroperoxide of 88% based on the cumene converted. The acid decomposition as in Example 1 leads to the formation of phenol with a yield of 84% with respect to the cumene converted.

### EXAMPLE 5

A solution of 50 mmoles of cumene, 5 mmoles of N-hydroxyphthalimide and 5 mmoles of acetaldehyde in 100 mL of acetonitrile is stirred at 20° C. for 24 hours in an oxygen atmosphere at atmospheric pressure. HPLC analysis shows a conversion of cumene of 68% with a yield to cumyl-hydroperoxide of 94% based on the cumene converted. 2 g of Amberlyst 15 are added to the solution and the mixture stirred at room temperature for 1 hour, leading to the formation of phenol with yields of 91% with respect to the cumene converted. The Amberlyst, insoluble in the reaction environment was separated and reused without loosing its catalytic activity.

### EXAMPLE 6

The same procedure is effected as in Example 5 without N-hydroxyphthalimide; there is no significant reaction.

### EXAMPLE 7

The same procedure is effected as in Example 5 without acetaldehyde; there is no significant reaction.

### EXAMPLE 8

The same procedure is effected as in Example 5 adding 0.1 mmoles of m-chloroperbenzoic acid during the reaction. The cumene conversion is 77% with a 93% yield to hydroperoxide based on the cumene converted and 89% to phenol after acid catalysis based on the cumene converted.

### EXAMPLE 9

The same procedure is effected as in Example 8 using benzaldehyde in the place of acetaldehyde. The cumene conversion is 59% with a 97% yield to hydroperoxide based on the cumene converted. The acid decomposition of the hydroperoxide leads to a yield of 92% to phenol based on the cumene converted.

### EXAMPLE 10

The same procedure is effected as in Example 8 using acetone as solvent instead of acetonitrile. The cumene conversion is 39% with a yield to hydroperoxide and phenol of 97% and 92% respectively based on the cumene converted.

### EXAMPLE 11

A solution of 5 mmoles of dimethyldioxirane in 10 mL of acetone is added dropwise under stirring to a solution of 50 mmoles of cumene and 2.5 mmoles of N-hydroxyphthalimide in 100 mL of acetone, at 20° C., in an oxygen atmosphere, at atmospheric pressure, over a period of 12 hours. The conversion of cumene is 45% with a yield to hydroperoxide of 97% based on the cumene converted. Decomposition by means of heterogeneous catalysis as in example 5 leads to a yield to phenol of 93% with respect to the cumene converted.

### EXAMPLE 12

The same procedure is effected as in Example 11 without N-hydroxyphthalimide; a conversion of 4% of cumene in cumyl alcohol is obtained.

### EXAMPLE 13

A solution of m-chloroperbenzoic acid (5 mmoles) in 10 mL of acetic acid are added dropwise under stirring to a solution of cumene (50 mmoles) and N-hydroxyphthalimde (5 mmoles) in 100 mL of acetic acid, over a period of 15 hours, under oxygen, at atmospheric pressure and 25° C. After decomposition with Amberlyst 15 according to Example 5, a cumene conversion of 62% is obtained with a yield of 89% to phenol with respect to the cumene converted.

### EXAMPLE 14

0.5 mmoles of m-chloroperbenzoic acid are added dropwise at 50° C., over a period of 24 hours, to a solution of 5 mmoles of cumene, 0.5 mmoles of N-hydroxysuccinimide in 10 mL of acetonitrile, in an oxygen atmosphere at ordinary pressure. A conversion of 45% is obtained with a yield to phenol of 88% with respect to the cumene converted.

1. A process for the preparation of cumene hydroperoxide wherein cumene is reacted with oxygen in the presence of a catalytic system selected from the group consisting of an N-hydroxyimide and an N-hydroxysulfonamide having general formula I or II, 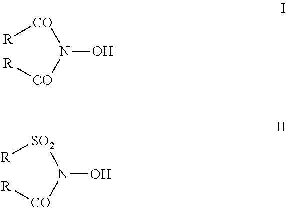
wherein R is an alkyl group, an aryl group or is part of aliphatic and aromatic cyclic system, associated with a peracid or dioxirane, at a temperature<100° C.

2. The process according to claim 1, wherein the N-hydroxyimide or N-hydroxysulfonamide is selected from the group consisting of N-hydroxysuccinimide, N-hydroxyphthalimide, and N-hydroxysaccharine.

3. The process according to claim 1, wherein the reaction is carried out at temperatures ranging from 20° C. to 70° C.

4. The process according to claim 1, wherein the reaction is carried out at atmospheric pressure.

5. The process according to claim 1, wherein the peracid is selected from aliphatic or aromatic peracids.

6. The process according to claim 5, wherein the peracid is selected from peracetic acid or m-chloroperbenzoic acid.

7. The process according to claim 1, wherein an aliphatic or aromatic aldehyde is used in the place of the peracid, which under the reaction conditions acts as a precursor of the peracid.

8. The process according to claim 7, wherein the aldehyde is selected from acetaldehyde or benzaldehyde.

9. The process according to claim 1, wherein the dioxirane is selected from aromatic and aliphatic dioxiranes.

10. The process according to claim 1, wherein a ketone and potassium monopersulfate are used in the place of the dioxirane, which under the reaction conditions act as a precursor of the dioxirane.

11. The process according to claim 1, wherein the peracid or dioxirane are added slowly to the reaction mixture.

12. The process according to claim 1, wherein the reaction is carried out in the presence of a solvent.

13. The process according to claim 1, wherein the quantity of N-hydroxy-derivatives, peracids or dioxiranes ranges from 1% to 10% with respect to the cumene.

14. The process according to claim 7, wherein when the N-hydroxy-derivative is associated with the aldehyde, the quantity of the latter ranges from 1% to 20% with respect to the cumene.

15. A process for the preparation of phenol which comprises the preparation of cumene hydroperoxide according to the process of claim 1 and the subsequent acid decomposition of the hydroperoxide to phenol and acetone.

16. The process according to claim 15, wherein the acid decomposition of the hydroperoxide takes place by means of heterogeneous acid catalysis in the presence of acid polymers selected from Amberlyst 15 or Nafion.

17. The process according to claim 15, wherein the acid decomposition of cumene hydroperoxide takes place by means of homogeneous acid catalysis.
